(19) United States
(12) Patent Application Publication
(10) Pub. No.: US 2012/0070594 A1
(43) Pub. Date: Mar. 22, 2012

(54) HYDROGENATED BISPHENOL-A-BASED POLYMERS AS SUBSTITUTES FOR BISPHENOL-A-BASED POLYMERS

(75) Inventors: William B. Carlson, Seattle, WA (US); Gregory D. Phelan, Cortland, NY (US); Phillip A. Sullivan, Seattle, WA (US)

(73) Assignee: EMPIRE TECHNOLOGY DEVELOPMENT LLC

(21) Appl. No.: 13/146,978

(22) PCT Filed: Sep. 17, 2010
(86) PCT No.: PCT/US10/49290

§ 371 Date: Jul. 29, 2011

(51) Int. Cl. B32B 1/08; C09D 169/00; C09D 151/08; C09D 175/04; C09D 167/02; A61L 31/10

(52) U.S. Cl. 428/35.7; 523/100; 524/604; 523/400; 524/540; 524/590; 524/612

(57) ABSTRACT

Compositions (including coatings) for food or beverage containers and medical devices, comprising a hydrogenated bisphenol-A-based polymer. Food or beverage containers and medical devices coated with hydrogenated bisphenol-A-based polymers. Food or beverage containers and medical devices made from hydrogenated bisphenol-A-based polymers.

### BACKGROUND

4,4′-(Propane-2,2-diyl)diphenol, more commonly known as bisphenol-A (BPA), is a widely used monomer for the production of polymers. The primary use of BPA is to create polymers including epoxy resins, polyurethanes, polyacrylates and polycarbonates. The aromatic groups of BPA are highly rigid, leading to polymers with great mechanical strength and high glass transition temperatures.

As a result, BPA-based polymers and resins are found in a wide range of products and applications, from consumer products to medical devices. For example, BPA-based epoxy resins are used for coil and can coatings for food and beverage containers; BPA-based polycarbonates and their copolymers are used to produce food containers including baby bottles, tableware, water bottles; and BPA-based polymers are used in medical devices including storage devices, renal dialysis devices, cardiac surgery products, surgical instruments, and intravenous connection components. Such widespread use has made BPA among the highest production volume industrial chemicals, leading to a substantial production infrastructure for the compound.

In recent years, health concerns have arisen regarding BPA-based polymers. Such polymers are susceptible to degradation and yellowing upon exposure to light, heat and certain chemicals. Upon degradation of the polymers, BPA and its derivatives can make its way into the contents of the food and beverage containers or medical storage devices and, subsequently, into the body. For example, BPA-containing polycarbonates have been shown to hydrolyze and release BPA monomers ((a) Mercea, P., Journal of Applied Polymer Science (2009), 112(2), 579; (b) Kang, Jeong-Hun; Kondo, Fusao. Food Additives & Contaminants (2002), 19(9), 886; (c) Howe, Susan R.; Borodinsky, Lester, Food Additives and Contaminants (1998), 15(3), 370; (d) Mountfort, Katrina A.; Kelly, Janet; Jickells, Sue M.; Castle, Laurence, Food Additives and Contaminants (1997), 14(6-7), 737). BPA is considered to be an endocrine disruptor and has been suggested to cause or contribute to birth defects, miscarriages, neurological problems, menstrual cycle disruptions, testicular disruption, and breast growth in males among other effects. In view of these concerns, various government authorities around the world have become more restrictive in regulating the amounts of BPA in certain products, and bans on the use of BPA in certain products such as baby bottles, have been instituted in some countries.

### SUMMARY OF THE TECHNOLOGY

As a replacement for BPA-based polymers, the present technology provides polymers containing hydrogenated BPA, i.e., 4-[2-(4-hydroxycyclohexyl)propan-2-yl]cyclohexan-1-ol (HBPA), and derivatives of HBPA. Such polymers and compositions containing such polymers can be inexpensive, easy to make by using current infrastructure, and have low toxicity. HBPA-containing polymers and compositions of the present technology exhibit hydrolytic stability, heat resistance and/or chemical resistance. Finally, HBPA-containing polymers and compositions of the present technology may be formulated for high flexibility and/or excellent adhesion. Thus, the present HBPA-containing polymers and compositions may be used in the manufacture of food and beverage containers and medical devices and in coatings for the same.

In accordance with one aspect, the present technology provides compositions that include a polymer having at least one repeating unit, wherein the repeating unit is a hydrogenated bisphenol A-containing unit and the polymer is selected from polycarbonate, an epoxy resin, an alkyd resin, a polyurethane, and a copolymer of any of the foregoing. The hydrogenated bisphenol A-containing unit may be substituted or unsubstituted. The compositions may be formulated for use as coating compositions for food or beverage containers or for medical devices. Alternatively, the food or beverage containers or the medical devices may comprise the present compositions in whole or part (e.g., certain surfaces of the containers or devices may include the present compositions).

In some embodiments of the present compositions, the polymer is an epoxy resin comprising a plurality of hydrogenated bisphenol-A containing units having the formula (I):

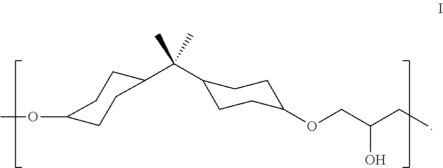

In some embodiments of the present compositions, the epoxy resin may be crosslinked. For example, the epoxy resin may be cross-linked by a polyamine, polyamide, polythiol, or polyol. In other embodiments, the polymer may be cross-linked by perfluorocyclobutane linkages.

In some embodiments of the present compositions, the polymer is a polyurethane. As a non-limiting example, the polyurethane may be cross-linked with an agent selected from the group consisting of methylene-bis(4-cyclohexylisocyanatc), 2,2-propylene-bis(4-cyclohexylisocyanate), isophorone diisocyante, hexamethylene diisocyante, hexamethylene diisocyanate dimer, hexamethylene diisocyanate trimer, polyisocyanates, toluene diisocyanate, methylene bis(4-phenylisocayante), benzene diisocyanate, and cyclohexane diisocyanate.

In other embodiments of the present compositions, the polymer is a polycarbonate comprising a plurality of hydrogenated bisphenol-A containing units having the formula (II):

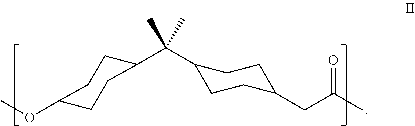

In some embodiments, the number of hydrogenated bisphenol-A containing units having the formula (II) ranges from 2 to 100,000.

The polycarbonate may further comprise a plurality of units derived from substituted or unsubstituted cyclohexane-based diols such as, e.g., a plurality of cyclohexyl units having the formula (III):

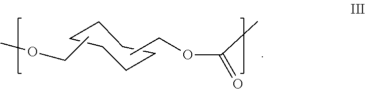

In some embodiments of the present compositions, the polycarbonate has the formula (V):

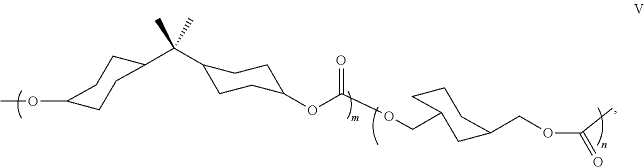

wherein m and n are independently from 2 to 100,000.

The polycarbonate may further comprise a plurality of units derived from substituted or unsubstituted aromatic diols. In some embodiments of the present compositions, the polycarbonate further comprises a plurality of bisphenol-A containing units having the formula (VI):

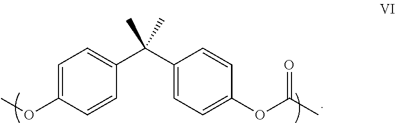

In other embodiments of the present compositions, the polycarbonate has the formula (VII):

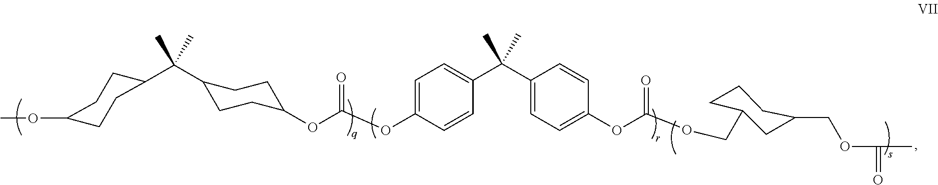

wherein q, r, and s are independently from 2 to 100,000.

In some embodiments of the present compositions, the polymer is an alkyd resin comprising a polyol backbone, comprising a plurality of hydrogenated bisphenol-A containing units; and a plurality of fatty acid side chain units attached to the polyol backbone. In an illustrative embodiment, the hydrogenated bisphenol-A containing unit has the formula (VIII):

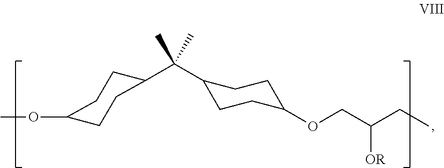

wherein R is H or a fatty acid side chain unit.

In some embodiments, the fatty acid side chain units comprise one or more of oleic acid, linolenic acid, linoleic acid, eleostearic acid, or palmitic acid. In some embodiments, the fatty acid side chain units comprise from about 80 to about 85% of eleostearic acid, from about 2 to about 6% of oleic acid, from about 3 to about 7% of palmitic acid, and from about 5 to about 10% of linoleic acid.

In some embodiments of the present compositions, the polymer is a random copolymer, a block copolymer or a graft copolymer. In some embodiments of the present compositions, the polymer is a graft copolymer wherein the backbone of the copolymer comprises at least one repeating unit selected from the group consisting of HBPA and HBPA-epichlorohydrin, and the graft side chain includes a polyacrylate or a polyolefin. In some embodiments, the graft side chain is a polyacrylate comprising at least one repeating unit selected from acrylic acid, acrylic acid esters, methacrylic acid and methacrylic acid esters. In other embodiments, the graft side chain is a polyolefin selected from the group consisting of polyethylene, polypropylene, polystyrene and a copolymer of any one thereof.

As noted above, the compositions described herein may be formulated for use as a coating for the surface of a food or beverage container or a medical device. Hence, in one aspect, the present technology provides food and beverage containers which include a surface coated with any of the compositions described herein, formulated for use as such a coating. In another aspect, the present technology provides medical devices which include a surface coated with any of the compositions described herein, formulated for use as such a coating.

Also as noted above, the compositions described herein may be formulated for use as a surface of a food or beverage container or a medical device. Thus, in another aspect, the present technology provides a food or beverage container comprising a surface, wherein the surface comprises such a composition. In another aspect, the present technology provides a medical device comprising a surface, wherein the surface comprises such a composition.

A medical device, comprising a surface, wherein the surface comprises a polymer having at least one repeating unit consisting of a hydrogenated bisphenol-A containing unit and the polymer is selected from a group consisting of polycarbonate, an epoxy resin, an alkyd resin, a polyurethane, and a copolymer of any of the foregoing.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 depicts an illustrative embodiment of the hydrogenation of BPA to HBPA.

FIG. 2 depicts illustrative embodiments of representative derivatives of hydrogenated BPA monomers

FIG. 3 depicts illustrative embodiments of representative diols and dicarboxylic acids for copolymerization with hydrogenated BPA monomers including cyclohexane-based diols and dicarboxylic acids, which are derived from the various isomers of xylene.

FIG. 4 depicts illustrative embodiments of representative monomers that can be copolyermized with HBPA-based monomers.

FIG. 5 schematically depicts illustrative embodiments of the synthesis of representative polycarbonates derived from HBPA-based monomers.

FIG. 6 depicts illustrative embodiments of representative non-toxic plasticizers that may be used to impart increased flexibility to hydrogenated bisphenol-A polymers. They are used at a concentration of 0.1-10%.

FIG. 7 schematically depicts an illustrative embodiment of the process of molding HBPA-based polymers into a food and beverage container of the desired shape.

FIG. 8 depicts an illustrative embodiment of a hydrogenation scheme for the surface of a solid BPA-based container.

FIG. 9 shows the calculations of the steric factor for hydrolytic stability of HBPA, neopentyl glycol, BPA, and ethylene glycol.

FIG. 10 depicts illustrative embodiments of the reactions of HBPA with ECH and the resulting structures.

FIG. 11 depicts representative fatty acids useful in creating HBPA-based alkyd resins.

FIG. 12 depicts illustrative embodiments of representative HBPA-based alkyds suitable for can coatings formed from tung oil and a hydroxyl polymer-based upon HBPA-epichlorohydrin prepolymer.

FIG. 13 is a schematic illustration of the placement of a pendent acrylic graft copolymer onto a HBPA containing backbone.

FIG. 14 is a schematic illustration of HBPA/epichlorohydrin polyol cross-linking with methylene-bis(4-cyclohexylisocyanate).

FIG. 15 shows the preparation of representative alkyd resins through fatty acid reaction with polyol.

FIG. 16 is a schematic illustration of the synthesis of representative alkyd resins C and D through the fatty acid reaction with glycidyl groups.

FIG. 17 is a schematic illustration of the synthesis of representative alkyd resins E and F of based upon a HBPA-epichlorohydrin polymer.

FIG. 18 shows the cross-linking of alkyd resins based upon HBPA and ricinoleic acid, which is derived from hydrogenated castor oil, with class I melamine formaldehyde resin as shown in the preparation of Coatings 3 and 4, Example 4.

FIG. 19 shows the cross-linking of alkyd resins based upon HBPA and oxidizing alkyd (linoleic acid) with HBPA dimethacrylate, as shown in the preparation of Coating 7 and 9, Example 4.

FIG. 20 shows the cross-linking of alkyd resins based upon HBPA and oxidizing alkyd (linoleic acid) with methylene-bis(4-cyclohexylisocyanate), as shown in the preparation of Coating 8, Example 4.

FIG. 21 shows a schematic process of placement of a pendent acrylic graft onto a backbone comprising of HBPA.

FIG. 22 illustrates the synthesis of cross-linked epoxy polymer as shown in Coating 16, Example 6.

FIG. 23 illustrates a representative 1K epoxy coating based upon a bis-imide and HBPA epoxy resin as shown in Coating 17, Example 6.

FIG. 24 illustrates a representative epoxy coating based upon a thiol and HBPA epoxy resin as shown in Coating 18, Example 6.

FIG. 25 illustrates a representative water borne HBPA diglycidyl ether epoxy system as outlined in Coating 19, Example 4.

FIG. 26 shows the synthesis scheme of polycarbonate 1, poly(hydrogenated 2-methyl-5-tert-butyl-BPA)carbonate, Example 8.

FIG. 27 shows the synthesis scheme of polycarbonate 2, poly(HBPA)carbonate, Example 8.

FIG. 28 shows the synthesis scheme of polycarbonate 3, poly(hydrogenated 2,5-dimethyl-bisphenol-A)carbonate, Example 8.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

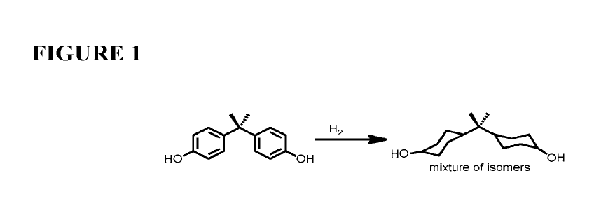

The present technology provides HBPA-based polymers as a substitute for BPA-based polymers in consumer products and medical applications. HBPA-based polymers may be prepared by hydrogenation through at least two representative methods. One method is to hydrogenate BPA monomers to form HBPA monomers (see, e.g., FIG. 1), which can then be polymerized into HBPA-based polymers. Such hydrogenations may be carried out with hydrogen and a transition metal catalyst such as Ni, Pt, or Pd. The other way is to polymerize the aromatic monomers into aromatic polymers. The aromatic polymers can then be hydrogenated to form aliphatic polymers using hydrogen with or without a catalyst. Representative catalysts include but are not limited to platinum, palladium, rhodium, ruthenium, and nickel based catalysts such as but not limited to Raney nickel and Urushibara nickel. The appropriate methods may be selected taking into account the cost effectiveness and desired structure of the final product(s).

HBPA-based polymers include at least one repeating unit that is a hydrogenated bisphenol A-containing unit. As used herein, the latter units are derived from both HBPA monomers and the hydrogenated products of substituted and unsubstituted BPA (collectively, “HBPA-based monomers”). Substituted BPA is BPA in which one or more hydrogens (e.g., 1, 2, 3, 4, 5, or 6 hydrogens) have been replaced with a non-hydrogen group and/or one or both methyl groups of BPA have been replaced with a non-methyl group (including, but not limited to hydrogen). In some embodiments, the substituents are selected from the group consisting of hydroxyl, halo (e.g., F, Cl, Br, I), alkyl, alkenyl, alkynyl, cycloalkyl, aryl (including phenyl), aralkyl, —COOH, alkoxy, aryloxy, aralkyloxy ester, thiol and sulfides, thioester, phosphines (including alkyl and aryl phosphines), amines (including alkylamines, and arylamines). The alkyl, alkenyl, alkynyl, cycloalkyl, aryl and aralkyl groups may be optionally substituted with hydroxyl or halo groups. In some embodiments, the hydrogenated BPA monomer has the formula below:

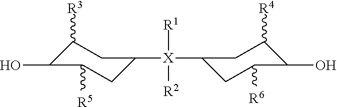

wherein

X is C; and

R¹-R⁶ are independently selected from the group consisting of H, OH, F, Cl, Br, I, alkyl, cycloalkyl, and phenyl groups, wherein the alkyl, cycloalkyl, and phenyl groups are optionally substituted with one or more substituents selected from the group consisting of OH, F, Cl, Br, and I; or

R¹ and R², together with X, may form a cycloalkyl group, optionally substituted with one or more halo groups.

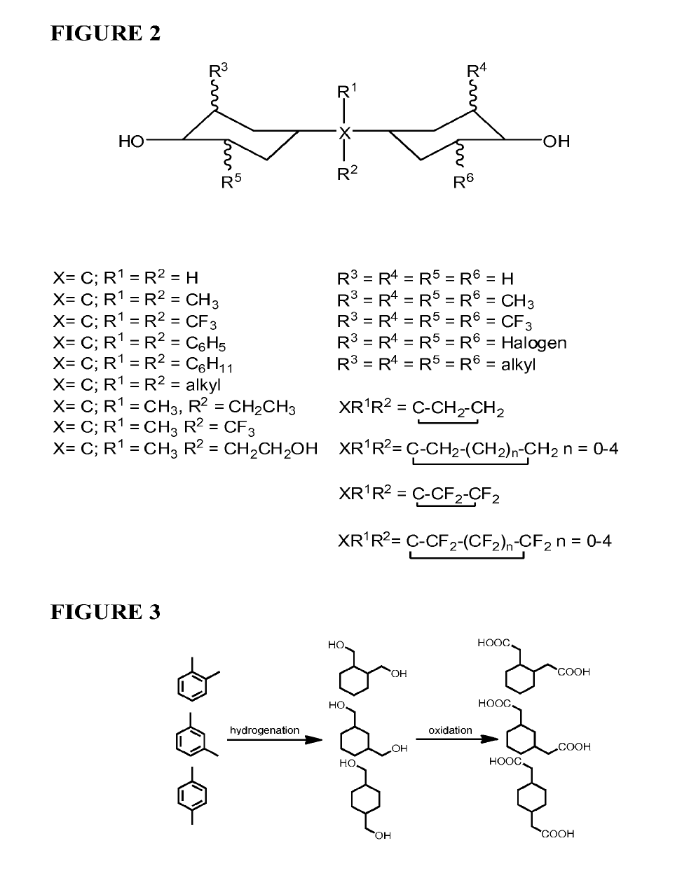

FIG. 2 illustrates a number of representative hydrogenated BPA monomers having the above formula.

Alkyl groups include straight chain and branched chain alkyl groups having 1 to 12 carbons or the number of carbons indicated herein. In some embodiments, an alkyl group has from 1 to 10 carbon atoms, from 1 to 8 carbons or, in some embodiments, from 1 to 6, or 1, 2, 3, 4 or 5 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups.

Cycloalkyl groups are cyclic alkyl groups having from 3 to 10 carbon atoms. In some embodiments, the cycloalkyl group has 3 to 7 ring members, whereas in other embodiments the number of ring carbon atoms range from 3 to 5, 3 to 6, or 5, 6 or 7. Cycloalkyl groups further include monocyclic, bicyclic and polycyclic ring systems. Monocyclic groups include, e.g., cyclopropyl, cyclobutyl, cyclopentyl, cyclohexyl, and cycloheptyl groups. Bicyclic and polycyclic cycloalkyl groups include bridged or fused rings, such as, but not limited to, bicyclo[3.2.1]octane, decalinyl, and the like. Cycloalkyl groups include rings that are substituted with straight or branched chain alkyl groups as defined above. Representative substituted alkenyl groups may be mono-substituted or substituted more than once, such as, but not limited to, mono-, di- or tri-substituted with substituents such as those listed above. Representative substituted alkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Thus, alkenyl groups have from 2 to 12 carbon atoms, and typically from 2 to 10 carbons or, in some embodiments, from 2 to 8, 2 to 6, or 2 to 4 carbon atoms. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Alkynyl groups include straight and branched chain alkyl groups as defined above, except that at least one triple bond exists between two carbon atoms. Thus, alkynyl groups have from 2 to 12 carbon atoms, and typically from 2 to 10 carbons or, in some embodiments, from 2 to 8, 2 to 6, or 2 to 4 carbon atoms. Examples include, but are not limited to —C≡CH, —CH≡CCH₃, —CH₂C≡CH, —CH(CH₃)C≡CH, —CH₂C≡CCH₃, —CH(CH₂CH₃)C≡CH, among others. Representative substituted alkynyl groups may be mono-substituted or substituted more than once, such as, but not limited to, mono-, di- or tri-substituted with substituents such as those listed above.

Aryl groups are cyclic aromatic hydrocarbons of 6 to 14 carbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain from 6 to 12 or even 6 to 10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups. Representative substituted aralkyl groups may be substituted one or more times with substituents such as those listed above.

Alkoxy groups are hydroxyl groups (—OH) in which the bond to the hydrogen atom is replaced by a bond to a carbon atom of an alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Representative substituted alkoxy groups may be substituted one or more times with substituents such as those listed above.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “ester” as used herein refers to —COOR³⁰ groups. R³⁰ is a substituted or unsubstituted alkyl, cycloalkyl, alkenyl, alkynyl, aryl, or aralkyl group as defined herein.

The term “amine” (or “amino”) as used herein refers to —NHR³⁵ and —NR³⁶R³⁷ groups, wherein R³⁵, R³⁶ and R³⁷ are independently hydrogen, or a substituted or unsubstituted alkyl, alkenyl, alkynyl, cycloalkyl, aryl or aralkyl group as defined herein. In some embodiments, the amine is NH₂, methylamino, dimethylamino, ethylamino, diethylamino, propylamino, isopropylamino, phenylamino, or benzylamino.

The term “thioester” as used herein refers to —C(O)SR⁴⁰ groups. R⁴⁰ is a substituted or unsubstituted alkyl, cycloalkyl, alkenyl, alkynyl, aryl, or aralkyl, group as defined herein.

The term “thiol” refers to —SH groups, while sulfides include —SR⁴¹ groups. R⁴¹ is a substituted or unsubstituted alkyl, cycloalkyl, alkenyl, alkynyl, aryl or aralkyl group as defined herein.

The term “phosphine” as used herein refers to —PR⁵⁰R⁵¹, wherein R⁵⁰ and R⁵¹ are independently selected from substituted or unsubstituted alkyl, cycloalkyl, alkenyl, alkynyl, aryl or aralkyl groups as defined herein.

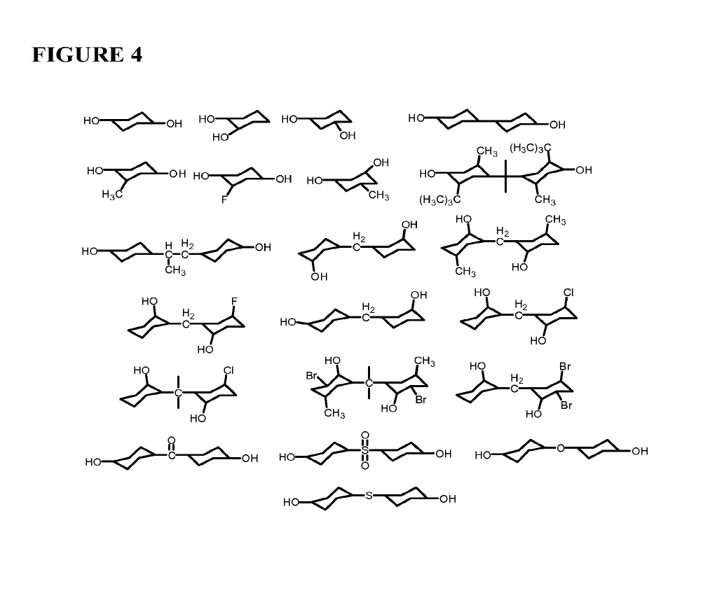

Other monomers, aromatic or aliphatic, may be copolymerized with the hydrogenated BPA monomers to provide the desired HBPA-based polymers. Aromatic monomers may include substituted and unsubstituted BPA. Aliphatic monomers may be obtained from common aromatic groups by hydrogenation. For example, common diols and dicarboxylic acids can be derived from various forms of xylene, as shown in FIG. 3. FIG. 4 illustrates representative monomers that can be copolymerized with hydrogenated BPA monomers.

While not wishing to be bound by theory, it is believed that the low toxicity of HBPA-based polymers is due to the lower toxicity of HBPA-based monomers. For example, the LD₅₀ (rat) value for HBPA is 5660 mg per kg; nearly half as toxic as BPA. The difference in toxicity of HBPA-based monomers compared to BPA-based monomers is likely because of the differences in structure and reactivity of the aliphatic alcohols and carboxylic acids compared to their aromatic counterparts. For example, the phenolic hydroxyl of BPA is much more acidic than aliphatic alcohols. The pKa for BPA is 9.6 while pKa for HBPA is 17. The difference leads to the different metabolic mechanisms for BPA and HBPA in the biological system, which likely would lead to less endocrine disrupting activity for HBPA when compared to BPA. The higher pKa of hydrogenated BPA monomers also leads to more hydrolytically stable polymers that will generally not leach monomer units into the contents of food and beverage containers or do so at a much reduced rate. Thus, HBPA and other similar cycloaliphatic diols and dicarboxylic acid are much less toxic than BPA, and result in safer, more durable polymers.

The HBPA-based monomers described herein can be copolymerized with similar or dissimilar monomers to form a variety of copolymers with properties tuned to specific applications. Representative HBPA-based polymers include polycarbonates, epoxy resins, alkyd resins, polyurethanes, and copolymers of any of the foregoing. The copolymers may be random copolymers, graft copolymers, or block copolymers. Polymers of the present technology may be cross-linked as described below. Compositions including HBPA-based polymers may be used in, for example, food and beverage containers and medical devices among others.

Polycarbonates. Polycarbonates are a class of condensation polymers that include multiple carbonate linkages. Polycarbonates of the present technology may be prepared from HBPA-based monomers using the same or similar techniques used in producing polycarbonates from BPA. HBPA-based monomers can be copolymerized with other non-HBPA-based cycloaliphatic monomers or aromatic monomers to provide polymers having a wide range of properties. For example, a variety of hydrolytically stable polycarbonates may be obtained according to the desired application by changing the types of monomer or the nature of the polymer, such as random or block copolymers. HBPA-based polymers may also be reinforced with fill materials known in the art such as fibers or inorganic particles if more rigid materials are needed.

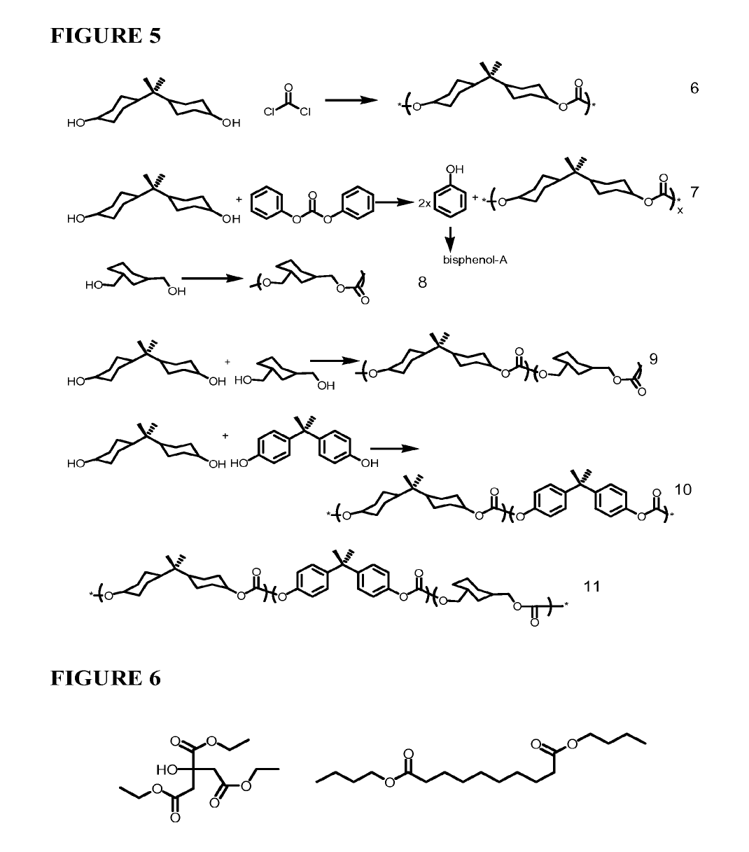

HBPA-based polycarbonates may be prepared by various routes, including, e.g., via the phosgene route (Goldberg, E. P., Polycarbonate resin compositions. (1964) U.S. Pat. No. 3,157,622) or through the transesterification route (Linear polycarbonates. (1965), FR 1391473) using diphenyl carbonate (see FIG. 5). Representative HBPA-based polymers include polycarbonates of HBPA (6, 7) cyclohexanedimethanol (8), or random and block copolymers of the two (9), as shown in FIG. 5. Polycarbonates may also be prepared from both HBPA-based monomers and other diols such as those listed in FIG. 4.

The HBPA-based polymers are more flexible than the aromatic counterparts. Unlike a BPA polycarbonate that is hard and brittle until plasticized, HBPA-based polycarbonates are tough, flexible materials even without plasticizers (Schnell, Hermann; Kimpel, Walter; Bottenbruch, Ludwig; Krimm, Heinrich; Fritz, Gerhard. Polycarbonate copolymers. (1957), DE 1011148). For example, poly(HBPA) carbonate is a tough rubbery material. Plasticizers may be used to impart additional flexibility and shock resistance to HBPA-based polycarbonates. FIG. 6 illustrates representative non-toxic plasticizers that may be used to impart increased flexibility to HBPA-based polymers. The plasticizers are used at a concentration of about 0.1 to about 10 weight percent (wt %) based on the total weight of the composition. In some embodiments, the plasticizer is used at a concentration of about 0.1 to about 5 wt %, about 1 wt % to about 10 wt %, about 1 wt % to about 8 wt %, about 2 to about 5 wt %. The resulting HBPA-based polymers may be used for flexible containers, bags, and tubing as food and beverage containers or in medical devices, among others.

Epoxy resins. BPA epoxy resins are often used as the interior linings of cans used for food and beverages. These resin systems have come under increasing scrutiny because the polymers may hydrolyze to produce BPA, which can leach ((a) Munguia-Lopez, E. M.; Peralta, E.; Gonzalez-Leon, Alberto; Vargas-Requena, Claudia; Soto-Valdez, Herlinda, Journal of Agricultural and Food Chemistry (2002), 50(25), 7299; (b) Munguia-Lopez, E. M.; Gerardo-Lugo, S.; Peralta, E.; Bolumen, S.; Soto-Valdez, H., Food Additives and Contaminants (2005), 22(9), 892; (c) Simoneau, C.; Theobald, A.; Hannaert, P.; Roncari, P.; Roncari, A.; Rudolph, T.; Anklam, E., Food Additives & Contaminants: Part A (1999), v16(5), 189; http://www.informaworld.com/smpp/title˜db=all˜content=t713599661˜tab=issueslist˜branches=16); (d) Kawamura, Y.; Sano, H.; Yamada, T. Journal of Food Hygiene Society Japan (1999), 40(158), 165; (e) Cao, X.-L.; Corriveau, J.; Popovic, S. Journal of Agricultural and Food Chemistry (2009) 57(4), 1307) into the consumable products and cause a multitude of health concerns. By contrast, HBPA-based polymers are much more hydrolytically stable ((a) Kim, Kyu-jun; Mochrie, Steve; Yang, Shi. (2004), WO 2004058892; (b) Henson, Walter A.; Helmreich, Robert F.; Johnson, Wilbur E. (1962), U.S. Pat. No. 3,061,559; (c) Hayes, B. T., SPE Transactions (1964), 4(2), 90). Thus, less HBPA will leach into food products, which leads to less toxicity and less endocrine disruptive behavior by BPA. These properties make HBPA epoxy resins especially suited for interior linings of food and beverage containers and coatings for medical storage devices.

Epoxy resins of the present technology may be prepared analogously to BPA-containing epoxy resins. For example, a polyol is formed from HBPA-based monomers and is then reacted with epichlorohydrin to impart epoxide functionality to the resin. Like BPA-containing counterparts, epoxy resins of the present technology crosslink through the epoxy groups. Representative functional groups of crosslinking agents that can react with epoxy groups include, but are not limited to, amines, amides, mercaptans, hydroxyl, and carbocations. In addition epoxy resins (or their polyol counterparts) may be crosslinked by perfluorocyclobutane (PFCB) linkages.

To form the perfluorocyclobutane linkages, the epoxy resin may be reacted with 1,2-dibromo-1,1,2,2-tetrafluoroethane and debrominated to form trifluorovinyl ethers on the resin and subjected to heat (e.g., about 220° C.). See, e.g., Choi, W.-S., Harris, F. W., Polymer (2000) 41(16), 6213. The crosslinked polymers obtained from trifluorovinylethers are hydrolytically stable and extremely solvent resistant. The added fluorine in the polymer lowers the surface energy and increases the hydrophobicity of the polymer. The prepolymers can be dispersed in water to create low VOC coatings. The water evaporates during the cure cycle.

FIG. 10 shows an illustrative embodiment of how HBPA-based monomers (here HBPA itself) can react with epichlorohydrin (ECH) to provide diglycidyl ethers, which may be reacted further to provide epoxy polymers. Similar chemistry may be performed using other cycloaliphatic diols. Referring to FIG. 10, HBPA acts as a nucleophile and opens the epoxy ring to form the initial epoxy substituted HBPA 1. The reaction may proceed in two pathways. HBPA 1 can react with ECH to from the bis epoxy adduct of HBPA, 2. HBPA 1 can also react with another HBPA monomer to form a hydroxyl terminated prepolymer 3. Further reactions lead to resins 4, of various molecular weights. As molecular weight increases, so does the chance of side reactions involving the interior hydroxyl groups, leading to branching Epoxy resins 5 form in the presence of an excess of ECH. Branching and functionalizing of the interior hydroxyl groups can also result from structure 5 with higher molecular weights and large excesses of ECH. Resulting epoxy resins of 5 are cross-linkable by a variety of hardeners as noted above.

In some embodiments, the weight average molecular weight of epoxy resins ranges from about 300 Daltons to about 1,000,000 Daltons. In some embodiments, the weight average molecular weight of epoxy resins ranges from about 380 Daltons to about 500 Daltons, from about 500 Daltons to about 5,000 Daltons, and from about 5,000 Daltons to about 15,000 Daltons. In some embodiments, the epoxy equivalent weight (EEW) ranges from about 150 to about 500, from about 190 to about 250, from about 250 to about 2,500, and from about 2,500 to about 8,000.

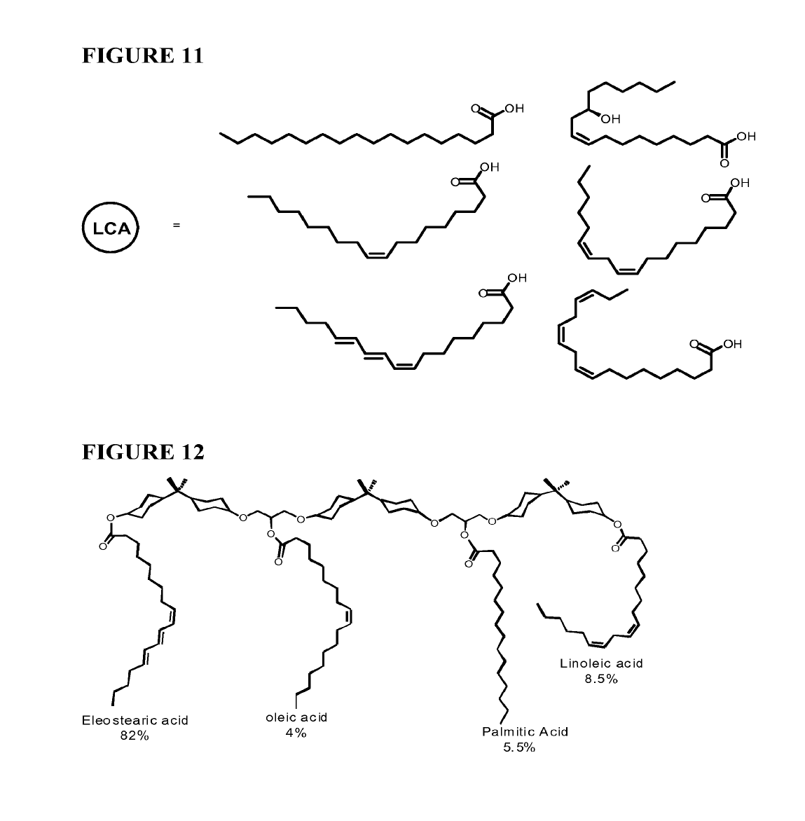

Alkyd resins. Alkyd resins of the present technology contain fatty acids and either HBPA-based monomers or an HBPA-based polymer. Fatty acids may be derived from natural oils such as tall oil, linseed oil, soybean oil, coconut oil, castor oil, sunflower oil, safflower oil, and tung oil. Depending on the oil type and composition, the saturated fatty acid contents vary in the range of 2.0 to 95.0 wt %, whereas the unsaturated fatty acid contents vary from 10.0 to 98.0 wt %. In some embodiments, the combination of fatty acids used to make the alkyd have an average number of methylene groups between double bonds greater than 2.0. In some embodiments, the various oils contain fatty acids having from 8 to 24 carbons, 10 to 20 carbons or 12 to 18 carbons in their carbon chains. In some embodiments the oils may contain saturated fatty acids with a C₈, C₁₀, C₁₄, C₁₆, and/or C₁₈ carbon chain. In an illustrative embodiment shown in FIG. 11, the saturated fatty acids content in the oils may be a mixture of lauric, stearic, and/or palmitic acids. In another embodiment, the unsaturated fatty acids in the oils may include oleic acid, linoleic, linolenic, ricinoleic, and/or eleostearic acids.

Fatty acids processed from the oil can be esterified with polyols to form alkyds. The fatty acid residue (i.e., the alkanoyl or alkenoyl) that is attached to a hydroxyl of the polyol is also referred to as a “fatty acid side chain unit” herein. The fatty acids (and therefore fatty acid side chain units) may be saturated (alkyl) or un-saturated (alkenyl) and may have from 8 to 24 carbons, 10 to 20 carbons or 12 to 18 carbons. The saturated fatty acids such as stearic acid are inert and act as plasticizers in the final polymer product. The unsaturated fatty acids such as oleic acid, linoleic acid, and linolenic acid provide a crosslinking mechanism to form high molecular weight thermosetting resins. The unsaturated fatty acids provide a different degree of reactivity and crosslinking ability and may be mixed in various ratios to tailor the properties of the crosslinked coating. Oleic acid leads to alkyds that have a low crosslink density while linolenic leads to alkyds that have a high crosslink density.

The polyol component of the alkyd resin includes HBPA-based monomers and polymers. The polyol component of the alky resin may also include glycidyl ethers of HBPA and related cycloaliphatics. Alkyds are particularly useful for water-based emulsion systems as the alkyd resins are easily dispersed in emulsion form and water does not interfere with the polymerization mechanism. They can also be made into high solids and even solvent free systems. Thus, low to zero VOC coating systems that are low in toxicity can be made using alkyd-based cycloaliphatic polymers.

In some embodiments, from 5 wt % to 75 wt % of tung oil, from 5 wt % to 75 wt % of linseed oil, or from 5 wt % to 75 wt % of castor oil is reacted with the polyol component to provide alkyd resins of the present technology. FIG. 12 shows a representative alkyd of the present technology suitable for can coatings. The alkyd of FIG. 12 is formed from tung oil and a polyol based upon HBPA-epichlorohydrin prepolymer. In some embodiments, the amount of fatty acid side chain units of the alkyd range from about 80 to about 85% of eleostearic acid, from about 2 to about 6% of oleic acid, from about 3 to about 7% of palmitic acid, and from about 5 to about 10% of linoleic acid by total weight of the side chains. Hydroxyl functional polyesters based upon HBPA and other cycloaliphatics may also be used. Such a resin has a high crosslink density due to the high concentration of eleostearic acid. In some embodiments, the weight average molecular weight of alkyd resins ranges from 700 Daltons to 1,000,000 Daltons, and from about 700 Daltons to about 10,000 Daltons.

Graft Copolymer resins. HBPA-based grafted polymer resins may be derived from HBPA and related cycloaliphatic resins. The HBPA-based grafted polymer resins may be based upon HBPA-epichlorohydrin epoxy polymers. Alternatively, the terminal groups may be other groups such as hydroxyl or hydrogen. In one embodiment, the HBPA-based grafted polymer resin includes a backbone of HBPA, HBPA-epichlorohydrin, or a polymer containing HBPA with a pendent side chain or graft. The pendent side chain or graft may contain a chain of vinylic monomer-based groups. Representative vinylic monomers include acrylics, styrenics, vinyl carboxylic acids (e.g., vinyl acetate), vinyl chloride, or other vinyl containing monomers.

The graft portion of the HBPA-based graft copolymer may be emplaced by a free radical initiator such as benzoyl peroxide. Other useful initiator classes include, but are not limited to, the azo class, peroxide class, and acyl peroxide class. The pendent side chain or graft may include a variety of monomer groups. The monomers are chosen to impart desirable properties on the graft copolymer. For example, glassy groups such as methyl methacrylate or styrene provide hardness to the resulting resin while rubbery groups such as butyl acrylate provide flexibility. The pendent side chain or graft may be crosslinkable. For example, the pendent side chain or graft may include monomer groups such as hydroxyethyl methacrylate to provide functionality for crosslinking the polymer.

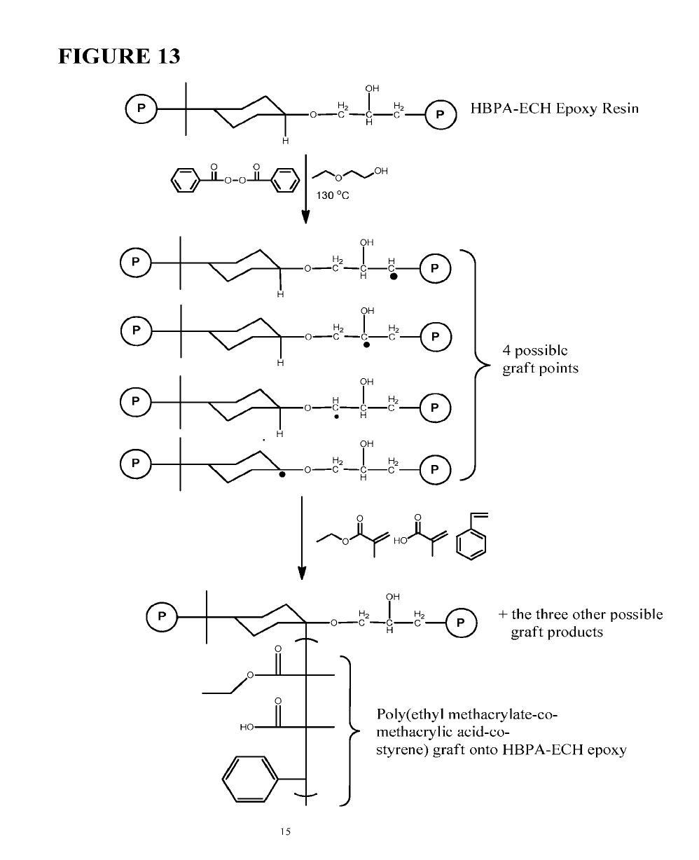

FIG. 13 shows a schematic of an illustrative embodiment of the placement of a pendent acrylic graft copolymer onto a HBPA containing backbone. HBPA-epichlorohydrin (HBPA-ECH) epoxy resin is used as the backbone. There are four possible graft points on HBPA-ECH epoxy backbone, which can lead to four possible graft products. FIG. 13 shows a representative HBPA-based graft copolymer, poly(ethyl methacrylate-co-methacrylic acid-co-styrene) grafted HBPA-ECH epoxy resin (15).

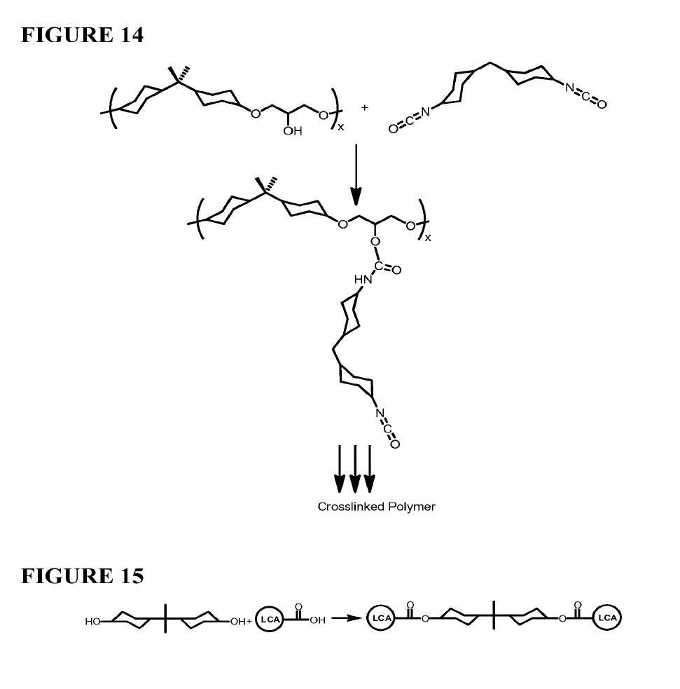

Polyurethane Coatings. Polyols of HBPA and epichlorohydrin can be cross-linked with isocyanates to form HBPA-based polyurethanes. These polyurethanes are hydrolytically stable. FIG. 14 shows an illustrative embodiment of HBPA/epichlorohydrin polyol cross-linking with methylene-bis(4-cyclohexylisocyanate). To form the HBPA-based polyurethane coatings, hydroxy-terminated HBPA-ECH resin may be combined with methylene-bis(4-cyclohexylisocyanate) and diluted with proper solvents, such as methyl acetate, t-butyl acetate, and p-chlorobenzotrifluoride. The solution is applied to substrate surface, such as tin coated steel, and heated to form a cross-linked polyurethane coating.

In another aspect, the present technology provides containers and devices made from HBPA-based polymers for food, beverage, and medical applications. The HBPA-based containers have the advantage of being non-endocrine disruptive and less toxic when compared to the BPA-based containers. Representative containers include medical vials, medical vials with attached septum as drug container, medical sample container, “Nalgene” bottles, food containers, baby bottles, beverage container, food storage containers, and plastic cups among others.

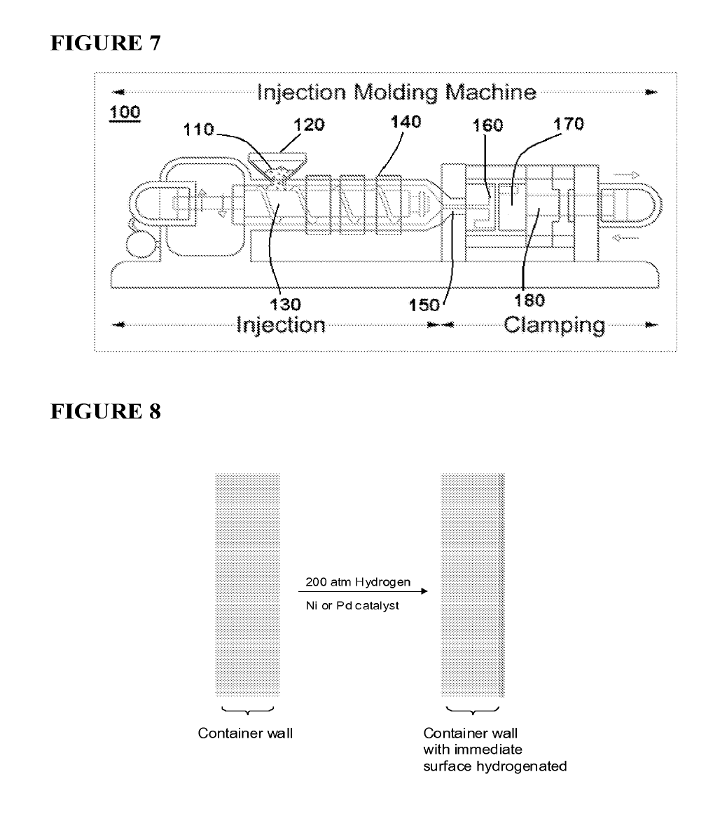

Rigid containers of HBPA-based polymers may be produced by an injection molding process. FIG. 7 is a schematic illustration of the process of molding HBPA-based polymers such as polycarbonates or polyesters into a food and beverage container of the desired shape. In an injection molding machine 100, the HBPA polymer 110 is added to a hopper 120 where it is carried by a screw 130. During the process, the polymer is heated to the melt temperature of 280° C. by the heater 140. The polymer is then injected into a mold cavity 160 through the nozzle 150. The mold of the container 170 is of the desired shape and size of the container. The formed container is then removed through a moveable platen 180.

Alternatively, the HBPA-based containers may be obtained by hydrogenation of solid containers made of BPA-based containers. In this process, the immediate surface, up to approximately 10 nm depth, of solid containers can be hydrogenated using a finely divided palladium or nickel catalyst. In one embodiment, as shown in FIG. 8, the container made of or coated by BPA is immersed in a solvent such as hexane in a hydrogenation container. The catalyst is introduced and the container pressurized to 250 atm of hydrogen. The system is heated to 150° C. and is agitated for 12 hours resulting in the hydrogenation of the immediate surface of the container. The containers are then washed extensively and the catalyst and solvents are collected to be reused.

In a further aspect, the present technology provides coating compositions for food and beverage containers and medical storage devices, comprising HBPA-based polymers. Representative HBPA-based polymers useful in the coating composition include epoxy resins, alkyd resins, water reducible graft copolymer coatings, and polyurethane coatings.

The compositions for the interior coating or lining of food and beverage containers may be dependent upon the food or beverage to be packaged in the container. According to the desirable property of a coating, the properties of HBPA-based polymers may be adjusted by using various known comonomers, either aliphatic or aromatic, and therefore may be used to substitute the BPA-based polymers in current coating processes.

The food or beverage is usually pasteurized at temperatures up to 120° C. for up to 60 minutes (potentially harsh conditions, especially when the food is acidic). Under these conditions, many BPA-based polymers can partially hydrolyze leading to leaching of BPA into the food or beverage contents. The HBPA-based polymers provide the advantage of high stability and low toxicity when compared to the traditional BPA-based materials used in the industry.

There are several polymer properties to consider in the design of interior coatings for beverage and food containers. Three very important properties are glass transition temperature, hydrolytic stability, and cross-link density. Aromatic groups are used because they are rigid and lead to polymers with a high glass transition temperature. The high glass temperatures leads to the use of plasticizers to soften polymers made with aromatic groups to make them more ductile and impart impact resistance. Straight chain aliphatic polymers typically have low glass transition temperatures and often do not require the use of plasticizers. The cycloaliphatic polymers, such as those made from HBPA, are in between the two extremes. The cyclic group imparts rigidity by the aliphatic nature of the ring allowing for increased molecular motion. Thus, while the cycloaliphatic polymers have lower glass transition temperatures than their aromatic counterparts, the glass transition temperature of the cycloaliphatics is substantially higher than the straight chain aliphatic polymers. Thus, HBPA-based cycloaliphatic polymers are more flexible and have greater impact resistance without (or with reduced) use of plasticizers than similar compositions utilizing aromatic groups.

Hydrolytic stability is a very important property for interior can coatings as the polymers are subject to aqueous environments at elevated temperatures for extended amounts of time. The issue of hydrolytic stability is one of great importance for BPA polycarbonates. Polycarbonate decomposes to BPA and carbon dioxide with repeated exposure to steam which in turn leads to a loss in polymer properties ((a) Pryde, C. A.; Kelleher, P. G.; Hellman, M. Y., Polym. Eng. Sci. (1982) 22, 370; (b) Hong, K. Z.; Qin, C.; Woo, L. Med. Plast. Biomat. (1996) May issue; (c) Asplund, B.; Sperens, J.; Mathisen, T.; Hilborn, J. J. Biomater. Sci., Poly. Ed. (2006) 17(6), 615; (d) Bair, H. E.; Falcone, D. R.; Hellman, M. Y.; Johnson, G. E.; Kelleher, P. G. (1981) J. App. Poly. Sci. 26(6), 1777). When polymers containing BPA are heated in a water environment, the monomer is ultimately released into the environment. The reason for this can be explained by the example of phenolic esters, which are structurally similar to carbonates. Esters formed with phenol (phenolic esters) are typically not hydrolytically stable due to specific chemical properties. The same phenomenon can be observed for phenyl methacrylate (a phenolic ester-based polymer). It is for this reason that, in the synthesis of polyesters, aromatic diacids are used with aliphatic alcohols and not the other way around.

The hydrolytic stability of a polymer can be estimated by Newman's “rule of six” steric factor (Equation 1). Specifically, the hydrolytic stability of polymer can be estimated by the number of atoms in the 6-position and then number of atoms in the 7-position. The higher the steric factor, the more hydrolytically stable the polymer would expect to be.

Steric factor=4(# of atoms in the six position)+(# of atoms in the 7 position) Equation 1

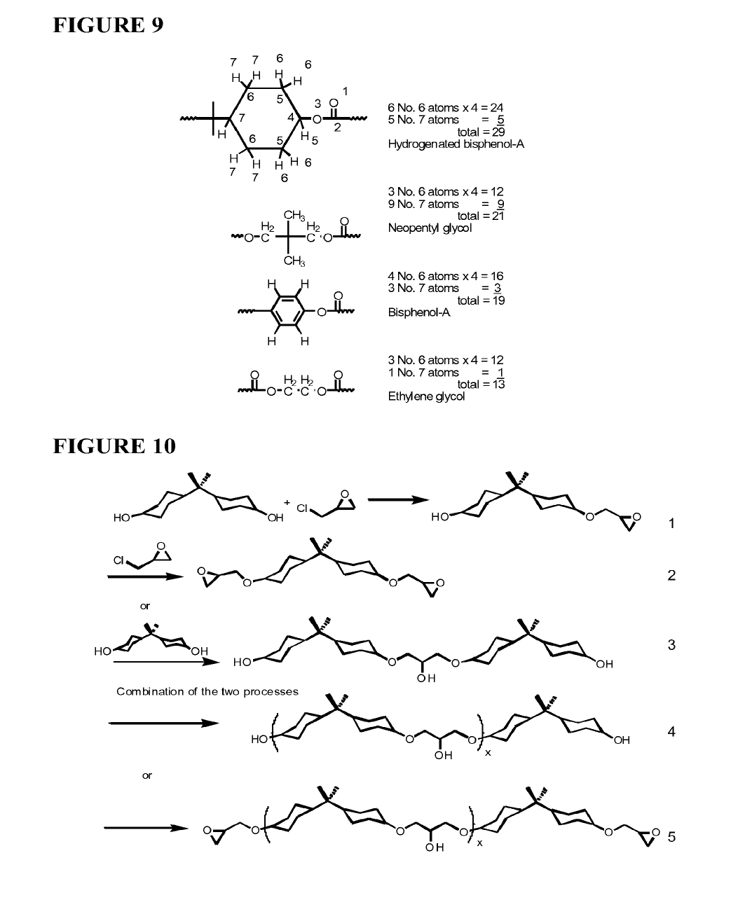

FIG. 9 shows calculation for HBPA, neopentylglycol, BPA, and ethylene glycol as an example. The Newman calculation in FIG. 9 predicts that HBPA-based polymers formed would be more hydrolytically stable than BPA-based polymers. However, cycloaliphatics such as HBPA or cyclohexanedimethanol are actually more resistant to hydrolysis than what the calculation of the steric factor suggests (Turpin, E. T. (1975) J. Paint. Technol. 47(602), 40). Thus, polymer based upon the cycloaliphatics, such as HBPA, would leach less monomer into the food medium. Therefore, toxicity of the polymers based upon the cycloaliphatics is reduced by lowering the amount of monomer contaminates in the food and reduced toxicity in the monomers themselves.

Cross-link density is important to the physical properties of the coatings. The cross-link density greatly impacts the solvent resistance, flexibility, hardness, and other properties of coatings. The property requirements for a specific use determine the right balance between all the properties in a polymer. Flexibility and hydrolysis resistance are two important properties for interior linings. Increased cross-link density often leads to increased resistance to solvents and hydrolysis but also leads to a decrease in flexibility.

### EXAMPLES

The present technology is further illustrated by the following examples, which should not be construed as limiting in any way.

### Example 1

### Preparation of Representative Alkyd Resins Through Fatty Acid Reaction with Polyol

Alkyd resins may be produced by mixing a fatty acid and the HBPA or HBPA polyol in a reaction vessel under nitrogen, as shown in FIG. 15. To the mixture is added 5% by weight xylene. A catalyst such as p-toluene sulfonic acid, tetraisopropyl titanate, lithium hydroxide, zirconium, zinc, calcium, ferrous, or lithium ricinoleate may be used. The catalyst may be removed after the completion of the reaction. A Dean-Stark trap is attached and the reaction vessel is heated to 230° C. The reaction is carried out until the acid value is below 5 mg KOH/g of resin.

Alkyd resin A. Alkyd resin A is prepared by reacting 120 g of HBPA (one equivalent of hydroxyl groups) with 283.3 g of linseed oil fatty acids.

Alkyd resin B. Alkyd resin B is prepared by reacting 120 g of HBPA with 300.4 g of hydrogenated castor oil fatty acids.

### Example 2

### Preparation of the Representative Alkyd Resins Through the Fatty Acid Reaction with Glycidyl Groups

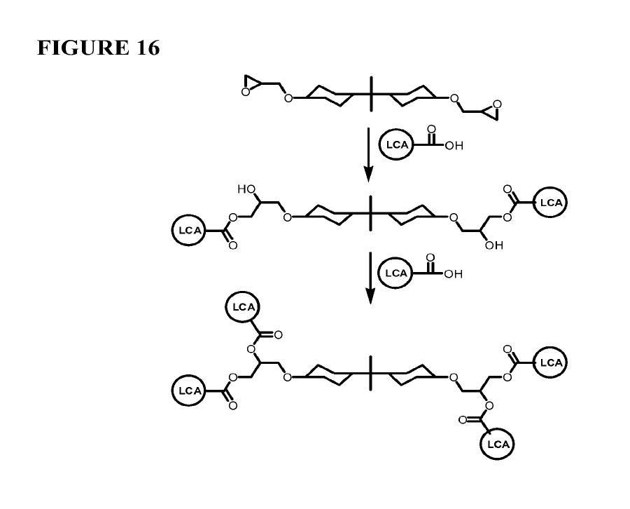

BPA diglycidyl ether with an epoxide equivalent weight of 1250 is dissolved into methylene chloride. BPA is dissolved into water with sodium hydroxide. In a two phase process, the BPA diglycidyl ether is reacted with the BPA sodium salt until the epoxy groups are consumed as measured by FT-IR. The water and methylene chloride fractions are separated and the methylene chloride layer is washed with additional water and then with 1 N HCl and is washed again with water. The hydroxy-terminated BPA-epichlorohydrin polymer is then hydrogenated. The synthesis is illustrated in FIG. 16.

Alkyd resin C is prepared by reacting 215 g of HBPA diglycidyl ether (SR-HBA) with 570 g of linseed oil fatty acids.

Alkyd resin D. Alkyd resin D is prepared by reacting 215 g of HBPA diglycidyl (SR-HBA) ether with 565 g of tung oil fatty acids.

### Example 3

### Preparation of Representative Alkyd Resins-Based Upon a HBPA-Epichlorohydrin Polymer

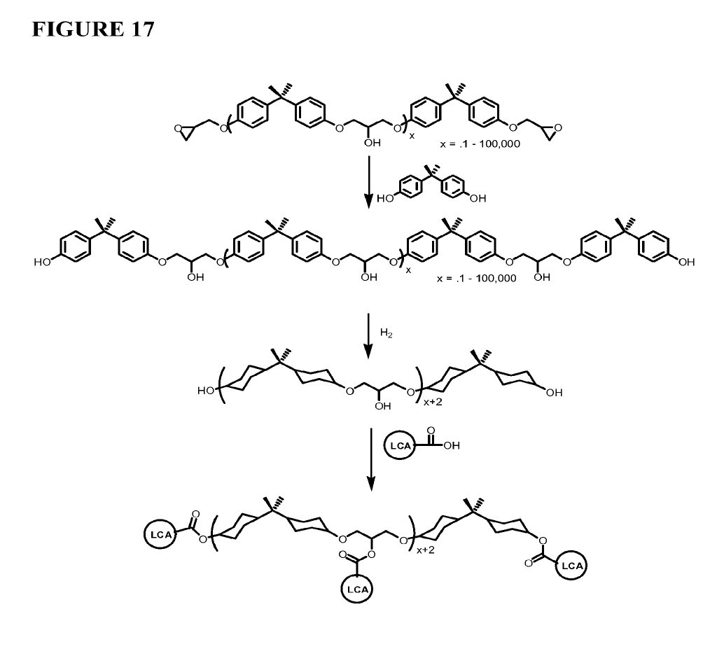

Alkyd resins may be based upon a HBPA-epichlorohydrin polymer, as shown in FIG. 17. This method prepares linear polymers of HBPA and epichlorohydrin. The alkyd resin can be prepared using glycidyl terminal groups as shown by alkyl resins C and D.

Alkyd resin E. Alkyd resin E is prepared by reacting 260 g of HBPA-epichlorohydrin resin (MW ˜2900) that is hydroxy-terminated (OH equivalent weight ˜260) with 287 g of linseed oil fatty acids.

Alkyd resin F. Alkyd resin F is prepared by reacting 260 g of HBPA-epichlorohydrin resin (MW ˜2900) that is hydroxy-terminated (OH equivalent weight ˜260) with 302 g of hydrogenated castor oil fatty acids.

### Example 4

### Preparation of Coatings Using the Representative Alkyd Resins

Coating 1. Alkyd resin A, 100 g, is measured into a 250 mL beaker. A catalytic mixture (drier) comprising of 13% zirconium octoate, 5% calcium octoate, and 1% cobalt octoate in mineral spirits is added to alkyd resin A carefully with stirring. The resin is applied to tin plated steel at a thickness of 2 mils and is baked at 120° C. for 30 minutes to form the film.

Coating 2. Alkyd resin A, 100 g, is measured into a 250 mL beaker. To alkyd resin A is added a mixture of zero VOC solvents: acetone, methyl acetate, t-butyl acetate, and p-chlorobenzotrifluoride. A catalytic mixture (drier) comprising 13% zirconium octoate, 5% calcium octoate, and 1% cobalt octoate in mineral spirits is added to alkyd resin A carefully with stirring. The resin is applied to tin plated steel at a thickness of 2 mils and baked at 120° C. for 30 minutes to form the film.

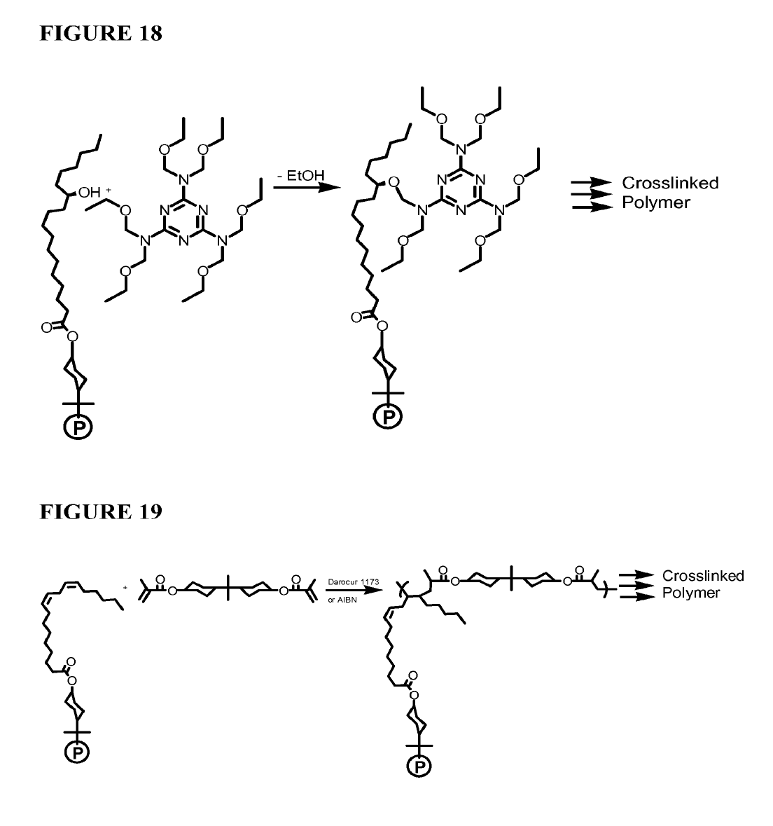

Coating 3. Alkyd resin B, 100 g, is measured into a 250 mL beaker. To alkyd resin B is added a mixture of zero VOC solvents: acetone, methyl acetate, t-butyl acetate, and p-chlorobenzotrifluoride. Class I melamine formaldehyde resin is added to the beaker and mixed. The mixture is sprayed onto tin plated steel at a thickness of 5 mils and is allowed to partially dry. The coating is then baked at 130° C. for 45 minutes to form the polymerized film. The synthetic scheme is outlined in FIG. 18.

Coating 4. Alkyd resin F, 105 g, is added to a 250 mL beaker. Alkyd resin B, 25 g, is added as a reactive diluent. To the alkyd mixture is added a mixture of zero VOC solvents: acetone, methyl acetate, t-butyl acetate, and p-chlorobenzotrifluoride. Class I melamine formaldehyde resin, 12.1 g, is added to the beaker and is mixed into the alkyd resins. The mixture is sprayed onto tin plated steel at a thickness of 5 mils and is allowed to partially dry. The coating is then baked at 130° C. for 45 minutes to form the polymerized film. The synthetic scheme is outlined in FIG. 18.

Coating 5. Alkyd resin E, 102 g, is measured into a 250 mL beaker. To alkyd resin E is added 10 g of alkyd resin A and 15 g of alkyd resin C as reactive diluents. To the alkyd mixture is added a mixture of zero VOC solvents: acetone, methyl acetate, t-butyl acetate, and p-chlorobenzotrifluoride. A catalytic mixture (drier) comprising of 13% zirconium octoate, 5% calcium octoate, and 1% cobalt octoate in mineral spirits is added (3.9 g) to alkyd resin carefully with stirring. The resin is applied to tin plated steel at a thickness of 4 mils and baked at 120° C. for 30 minutes to form the film.

Coating 6. Similar procedure as Coating 2, but with alkyd resin D.

Coating 7. Alkyd resin E, 107.2 g, is measured into a 250 mL beaker. To alkyd resin E is added 26.6 g of alkyd resin B. To the alkyd mixture is added a mixture of zero

VOC solvents: methyl acetate, t-butyl acetate, and p-chlorobenzotrifluoride. HBPA dimethacrylate, 25 g, is added and mixed in until completely dissolved. Darocure 1173 photo-initiator is then added dropwise with stirring and is completely mixed. The resin is applied to tin plated steel at a thickness of 4 mils, allowed to partially dry, and then heated to 50° C. under nitrogen. The sample is then, while under nitrogen, irradiated with UV light for 2 minutes to form the polymerized film. The synthetic scheme is described in FIG. 19. Other acrylics such as methyl methacrylate may be used as reactive diluents. The polymerization may be initiated by thermal means with a thermal initiator such as azobisisobutyronitrile or with UV light with a photo-initiator such as Darocur 1173.

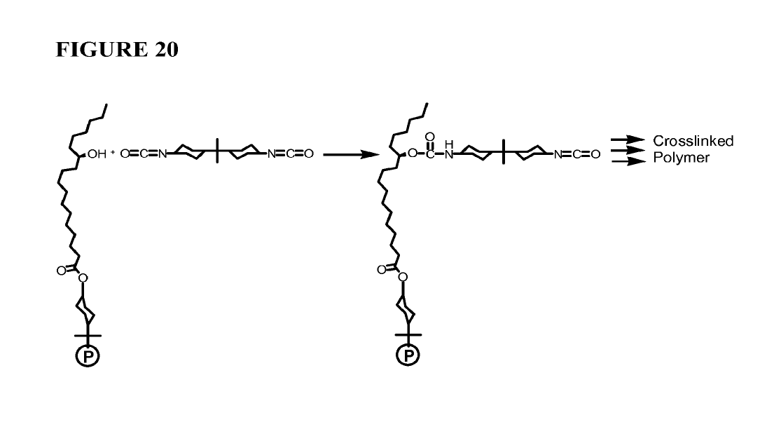

Coating 8. As shown in FIG. 20, alkyd resin F, 105 g, is added to a 250 mL beaker. Alkyd resin B, 25 g, is added as a reactive diluent. To the alkyd mixture is added a mixture of zero VOC solvents: t-butyl acetate, and p-chlorobenzotrifluoride. Methylene-bis(4-cyclohexylisocyanate), 30.1 g, warmed to 25-30° C. is added to the beaker and mixed into the alkyd resins. The mixture is sprayed onto tin plated steel at a thickness of 3 mils and allowed to partially dry. The coating is then baked at 130° C. for 30 minutes to form the polymerized film.

Coating 9. Alkyd resin E, 102.7 g, is measured into a 250 mL beaker. To alkyd resin E is added 10.0 g of alkyd resin A and 15.3 g of alkyd resin C. To the alkyd mixture is added a mixture of zero VOC solvents: t-butyl acetate, and p-chlorobenzotrifluoride. HBPA dimethacrylate, 24.7 g, is added and mixed in until completely dissolved. A solution of azobisisobutyronitrile in methyl acetate is made and is then added dropwise with stirring and is completely mixed. The resin is applied to tin plated steel at a thickness of 4 mils, allowed to partially dry, and then heated to 80° C. under nitrogen for four hours. The synthetic scheme is described in FIG. 19. Other acrylics such as methyl methacrylate may be used as reactive diluents. The polymerization may be initiated by thermal means with a thermal initiator such as azobisisobutyronitrile or with UV light with a photo-initiator such as Darocur 1173.

Coating 10. Similar procedure as Coating 1, but with a mixture of alkyd resins D and E.

The alkyd resins based upon HBPA are surprisingly resistant to hydrolysis. For example, it will be found that Coating A and B exhibit very little change in polymer properties even after long term immersion tests ASTM D870-09. The coating formation compositions for Coatings 1-5 are listed in Table 1. The coating formation compositions for Coating 6-10 are listed in Table 2.

TABLE 1
Coating formation compositions.

| | Coating | Coating | Coating | Coating | Coating |
|---|---|---|---|---|---|
| Ingredient^{a} | 1 | 2 | 3 | 4 | 5 |
| Alkyd A | 100.0 | 100.0 | — | — | 10.0 |
| Alkyd B | — | — | 100.0 | 25.0 | — |
| Alkyd C | — | — | — | — | 15.0 |
| Alkyd D | — | — | — | — | — |
| Alkyd E | — | — | — | — | 102.0 |
| Alkyd F | — | — | — | 105.0 | — |
| Acetone | — | 1.1 | 1.0 | 4.0 | 3.8 |
| Methyl acetate | — | 2.0 | 1.3 | 6.3 | 6.2 |
| t-butyl acetate | — | 4.9 | 4.8 | 10.9 | 11.0 |
| p-chlorobenzotrifluoride | — | 2.1 | 2.5 | 3.8 | 4.0 |
| Melamine formaldehyde^{b} | — | — | 16.0 | 14.5 | — |
| Drier^{c} | 2.0 | 2.0 | — | — | 3.9 |

^{a}parts by weight
^{b}as the ethyl ether
^{c}13% zirconium octoate, 5% calcium octoate, 1% cobalt octoate in mineral spirits

TABLE 2
Coating formation compositions.

| | Coating | Coating | Coating | Coating | Coating |
|---|---|---|---|---|---|
| Ingredient^{a} | 6 | 7 | 8 | 9 | 10 |
| Alkyd A | 9.8 | 10.1 | — | 10.0 | — |
| Alkyd B | — | — | 25.6 | — | — |
| Alkyd C | — | — | | | — |
| Alkyd D | 21.3 | 15.6 | — | — | 25 |
| Alkyd E | — | 103.1 | — | 102.7 | 100 |
| Alkyd F | — | — | 107.2 | — | — |
| Acetone | 0.3 | — | — | — | — |
| Methyl acetate | 0.5 | 9.7 | — | 8.7 | — |
| t-butyl acetate | 1.0 | 10.2 | 8.2 | 11.3 | — |
| p- | 0.7 | 6.0 | 5.3 | 5.0 | — |
| chlorobenzotrifluoride | | | | | |
| HBPA dimethacrylate | — | 25.0 | — | 24.7 | — |
| Methylene-bis(4- | — | — | 30.1 | — | — |
| cyclohexylisocyanate) | | | | | |
| Darocur 1173 | — | 0.5 | — | — | — |
| azobisisobutyronitrile | — | — | — | 2.1 | — |
| Drier | 0.9 | — | — | — | 3.0 |

^{a}parts by weight
^{b}13% zirconium octoate, 5% calcium octoate, 1% cobalt octoate in mineral spirits

### Example 5

### Preparation of a Representative Coating Using a Graft Copolymer Containing HBPA

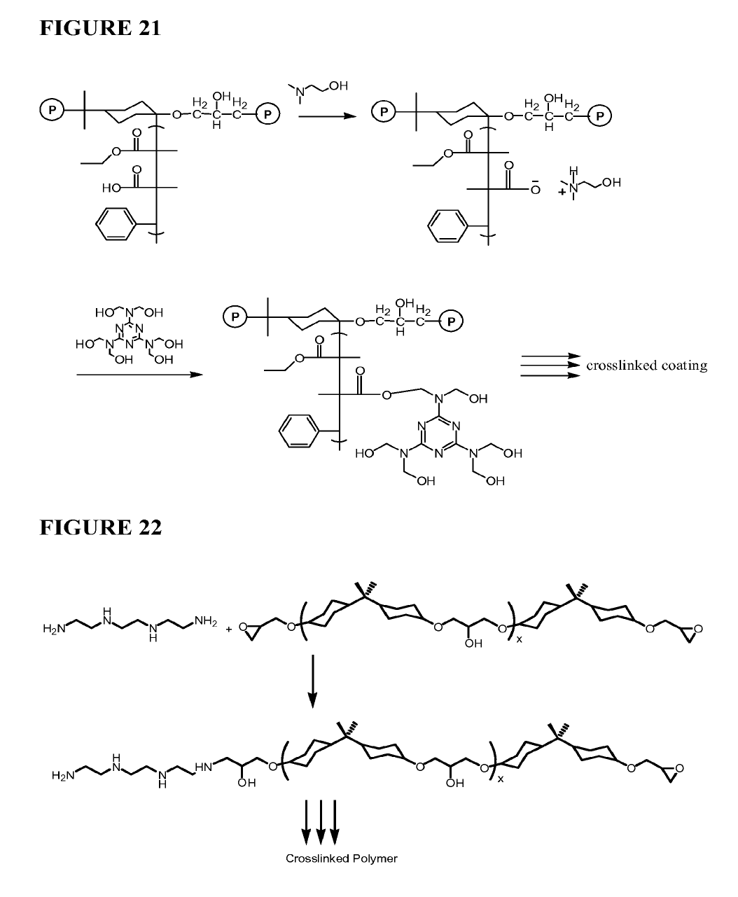

HBPA-epichlorohydrin resin (SR-HBA, epoxide equivalent weight 215) is reacted with BPA and is then hydrogenated. HBPA also can be used to extend SR-HBA but creates a branched polymer rather than linear. The extended SR-HBA resin is then dissolved into ethyl cellosolve. The solution is heated to 120° C. under nitrogen. To the HBPA/epichlorohydrin resin is added a solution of benzoyl peroxide, and the monomers such as ethyl methacrylate, methacrylic acid, and styrene at 0.1% wt. % SR-HBA to 1000 wt % SR-HBA by dropwise addition. The solution is heated to 130° C. under nitrogen and after addition the temperature is held for 2 hours. The resulting resin is a mixture of grafted acrylic resin on the HBPA-epichlorohydrin polymer backbone, un-grafted acrylic resin, and un-reacted SR-HBA. The graft points are on the alpha-hydrogen atoms to the oxygen on the epichlorohydrin portion of the SR-HBA as has been previously reported ((a) J. T. K. Woo, V. Ting, J. Evans, R. Marcinko, G. Carlson, C. Ortiz. J. Coat. Technol. (1982) 54, 689; (b) J. T. K. Woo, A. Toman Polym. Mater. Sci. Eng. (1991) 65, 323). Surprisingly, it is or will be found that grafting also takes place on the 4^{th} position on the cyclohexyl ring. The grafting onto this position helps prevent hydrolysis of the HBPA into solution. The synthetic scheme for the placement of a pendent acrylic graft polymer onto a backbone comprising of HBPA is illustrated in FIG. 21.

The solution is then cooled to 95° C. A blend of 2-(dimethylamino)ethanol and water is added dropwise. The acrylic grafted HBPA-epichlorohydrin resin is neutralized by 2-(dimethylamino)ethanol. De-ionized water is then added. The acrylic grafted HBPA-epichlorohydrin resin can be cross-linked by a variety of compounds such as melamine-formaldehyde resins, epoxy resins, alcohols or itself. The resulting dispersion can then be spray applied. The coating is baked to remove solvents and cross-link the polymer. Baking temperatures depend upon the cross-linker used. Cross-linking with melamine-formaldehyde resins can take place at 120° C. Cross-linking with epoxy resins (HBPA diglycidyl ether) takes place at 150° C. Cross-linking the polymer itself takes place at 180° C.

TABLE 3
Examples of grafted HBPA formulations.

| | Coating | Coating | Coating | Coating | Coating |
|---|---|---|---|---|---|
| Ingredient^{a} | 11 | 12 | 13 | 14 | 15 |
| SR-HBA^{b} | 208.0 | 210.2 | 209.1 | 207.8 | 209.5 |
| BPA^{c} | 110.2 | 111.3 | 110.8 | 110.0 | 111.1 |
| Catalyst 1201 | 0.3 | 0.3 | 0.3 | 0.3 | 0.3 |
| Xylene | 6.3 | 6.4 | 6.2 | 6.6 | 6.2 |
| Methacrylic acid | 25.3 | 26.1 | 25.9 | 26.8 | 26.5 |
| Ethyl methacrylate | — | — | 21.9 | — | — |
| Butyl methacrylate | — | — | — | 22.0 | — |
| Butyl acrylate | 22.1 | 22.3 | — | — | 22.3 |
| Hydroxylethyl | — | — | — | 3.7 | 3.6 |
| methacrylate | | | | | |
| Styrene | — | 19.1 | 19.4 | 19.2 | — |
| Methyl methacrylate | 19.9 | — | — | — | 19.8 |
| Benzoyl Peroxide | 2.6 | 2.6 | 2.6 | 2.6 | 2.6 |
| 2-(dimethylamino)- | 37.9 | 38.7 | 38.2 | 39.2 | 38.8 |
| ethanol | | | | | |
| Deionized water | 430.7 | 427.8 | 430.5 | 430.0 | 429.6 |

^{a}parts by weight
^{b}commercially available HBPA epoxy resin has EEW of 215.
^{c}BPA is used to extend the SR-HBA chain in a linear fashion and is then hydrogenated to remove all aromatic groups.

### Example 6

### Preparation of a Representative Epoxy Coating Containing Hbpa

HBPA-based epoxy coating may be obtained by the reaction of glycidyl groups derived from HBPA and related cycloaliphatic resins. The epoxy resins may be based upon HBPA-epichlorohydrin epoxy polymers and may be cured by a variety of groups. Examples of curing groups include amines, thiols, carboxylic acids (as shown in the alkyd section), phosphines, phenols, and alcohols.

Coating 16. As shown in FIG. 22, HBPA diglycidyl ether (107.0 g) with an EEW of 215 is diluted with methyl acetate (7.1 g), t-butyl acetate (10.0 g), and p-chlorobenzotrifluoride (7.9 g). To this mixture is added 31.5 g of triethylene tetraamine The solution is allowed to stand (induct) for 20 minutes. The solution is then sprayed onto tin coated steel plates and heated to 90° C. for 15 minutes.

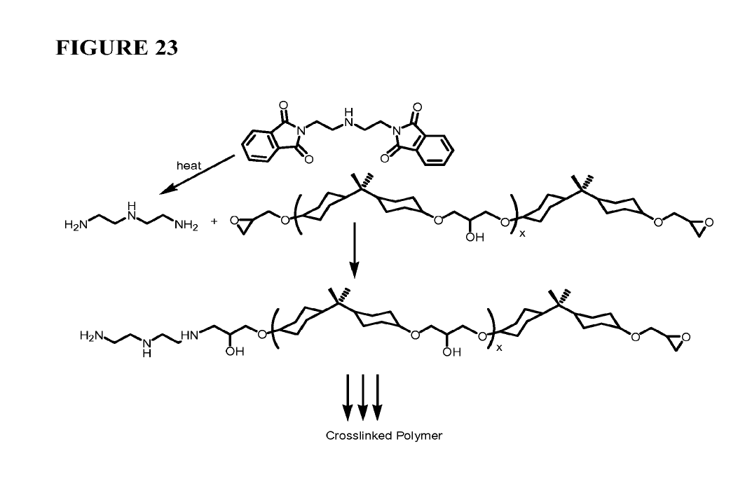

Coating 17. As shown in FIG. 23, HBPA diglycidyl ether (107.8 g) with an EEW of 215 is mixed with 78.0 g of 200 to about 400 mesh particle size bis-imide of diethylene triamine. The components are blended together. The solution is stable for six months. The solution can then be coated onto tin coated steel and heated to 120° C. for 30 minutes. The solution can be diluted with methyl acetate (15.1 g), t-butyl acetate (24.0 g), and p-chlorobenzotrifluoride (10.9 g) for spraying. The solution can then be sprayed onto tin coated steel and heated to 120° C. for 30 minutes.

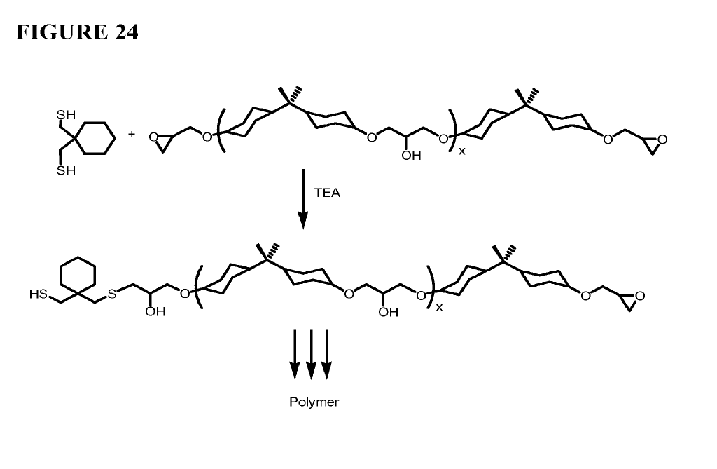

Coating 18. As shown in FIG. 24, HBPA diglycidyl ether (108.0 g) with an EEW of 215 is diluted with methyl acetate (10.0 g), t-butyl acetate (13.0 g), and p-chlorobenzotrifluoride (9.9 g). To this mixture is added 44.0 g of 1,1-bis(mercaptomethyl)cyclohexane and then 1.0 g of triethylamine. The solution is allowed to stand (induct) for 20 minutes. The solution is then sprayed onto tin coated steel plates and heated to 130° C. for 25 minutes.

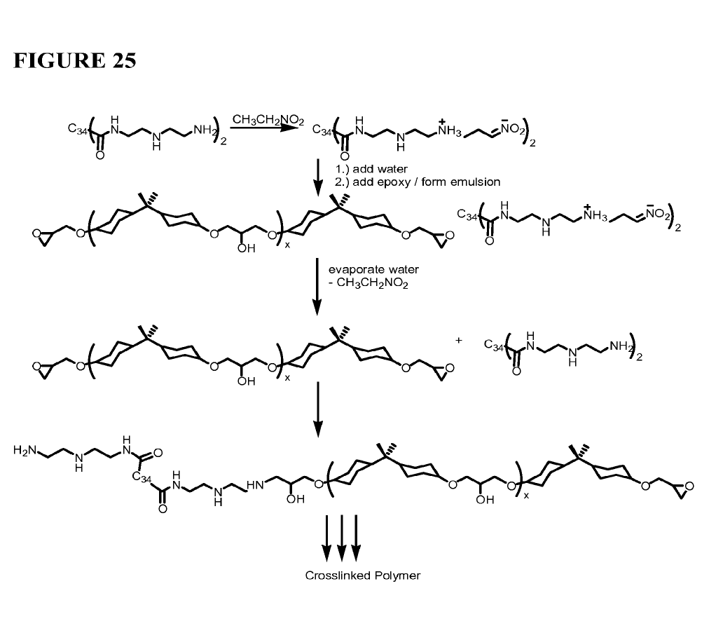

Coating 19. As shown in FIG. 25, HBPA diglycidyl ether (108.0 g) with an EEW of 215 is diluted with 25 g of 1-butanol. An amine resin (136 g) of the empirical formula C₃₄(C(═O)—NH—CH₂—CH₂—NH—CH₂CH₂—NH₂)₂ is reacted with ethyl nitrate to form the amine salt. Water, 250 g, is added to the amine salt. The HBPA diglycidyl ether/butanol solution is then added to the water/amine solution slowly with stirring. The butanol is then removed by reduced pressure. The water borne epoxy is then either spread or spray applied to tin-coated steel. The system is heated to 100° C. for 30 minutes to polymerize the coating.

### Example 7

### Preparation of a Representative Polyurethane Coatings Containing HBPA

Preparation of a representative polyurethane coating is shown in FIG. 14. Polyols of HBPA and epichlorohydrin can be cross-linked with isocyanates to form urethanes. Surprisingly these urethanes are hydrolytically stable. HBPA-epichlorohydrin resin (MW ˜3000) that is hydroxy-terminated (65 g) is combined with 8 g of methylene-bis(4-cyclohexylisocyanate) and diluted with methyl acetate (2.8 g), t-butyl acetate (6.0 g), and p-chlorobenzotrifluoride (3.2 g). The solution is applied to tin coated steel and heated to 120° C. for 20 minutes to form the cross-linked coating.

### Example 8

### Preparation of a Representative Polycarbonate Containing Hbpa

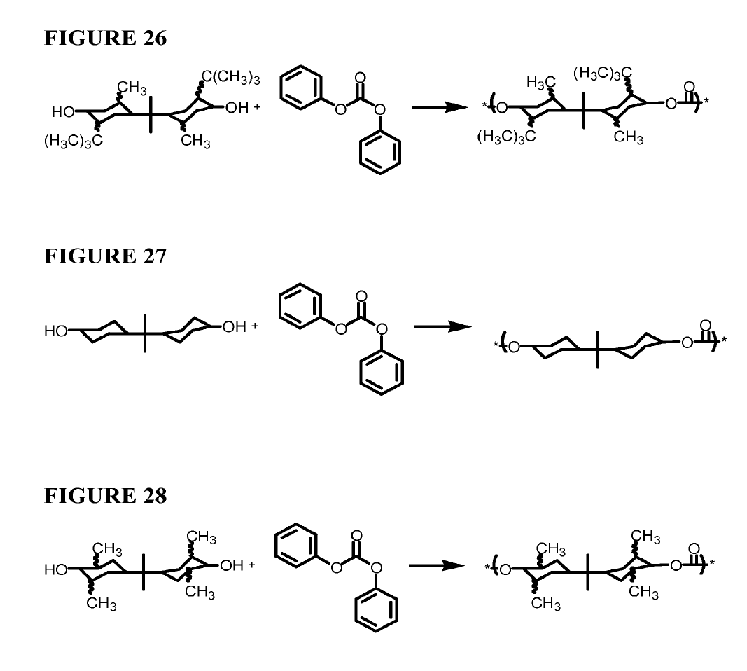

Polycarbonate 1. 380.65 g (1.00 mol) of hydrogenated 2-methyl-5-tert-butyl-bisphenol-A and 224.9 g (1.05 mol) of diphenyl carbonate are added into a three-necked glass reactor equipped with a mechanical stirrer, nitrogen inlet and a distillation system. In the first stage of the reaction the molten mixture is heated to 200° C. (as the internal temperature) and 0.5 g zinc acetate-2-hydrate (extra pure) is added. The pressure in the reactor is reduced to 20 mm Hg after 25 minutes to remove phenol, while the temperature is increased from 200 to 260° C. within 60 min; then the reactor pressure is further reduced to 1 mm Hg and the reaction is completed by heating for an additional 30 min at 260° C. under vacuum. The final polymer melt is cooled under vacuum to room temperature, then optionally dissolved in chloroform and precipitated dropwise from the solution into methanol. The poly(hydrogenated 2-methyl-5-tert-butyl-bisphenol-A)carbonate that will be obtained is dried under reduced pressure at 100° C. overnight. The polymer is ready to be melted and injection molded. The synthetic scheme is shown in FIG. 26.

Polycarbonate 2. 240.4 g (1.00 mol) of HBPA and 224.9 g (1.05 mol) of diphenyl carbonate are added into a three-necked glass reactor equipped with a mechanical stirrer, nitrogen inlet and a distillation system. In the first stage of the reaction the molten mixture is heated to 200° C. (as the internal temperature) and 0.3 g zinc acetate-2-hydrate (extra pure) is added. The pressure in the reactor is reduced to 20 mm Hg after 25 minutes to remove phenol, while the temperature is increased from 200 to 260° C. within 60 min; then the reactor pressure is further reduced to 1 mm Hg and the reaction is completed by heating for additional 30 min at 260° C. under vacuum. The final polymer melt is cooled under vacuum to room temperature, then optionally dissolved in chloroform and precipitated dropwise from the solution into methanol. The poly(HBPA)carbonate that will be obtained is dried under reduced pressure at 100° C. overnight. The polymer is ready to be melted and injection molded. The synthetic scheme is shown in FIG. 27.

Polycarbonate 3. 296.5 g (1.00 mol) of hydrogenated 3,5-dimethyl-bisphenol-A and 224.9 g (1.05 mol) of diphenyl carbonate are added into a three-necked glass reactor equipped with a mechanical stirrer, nitrogen inlet and a distillation system. In the first stage of the reaction the molten mixture is heated to 200° C. (as the internal temperature) and 0.3 g zinc acetate-2-hydrate (extra pure) is added. The pressure in the reactor is reduced to 20 mm Hg after 25 minutes to remove phenol, while the temperature is increased from 200 to 260° C. within 60 min; then the reactor pressure is further reduced to 1 mm Hg and the reaction is completed by heating for an additional 30 min at 260° C. under vacuum. The final polymer melt is cooled under vacuum to room temperature, then optionally dissolved in chloroform and precipitated dropwise from the solution into methanol. The poly(hydrogenated 3,5-dimethyl-bisphenol-A)carbonate obtained is dried under reduced pressure at 100° C. overnight. The polymer is ready to be melted and injection molded. The synthetic scheme is shown in FIG. 28.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A composition comprising a polymer having at least one repeating unit, wherein the repeating unit is a hydrogenated bisphenol A-containing unit and the polymer is selected from a group consisting of a polycarbonate, an epoxy resin, an alkyd resin, a polyurethane, and a copolymer of any of the foregoing, and further wherein the composition is a surface of or a coating for a food or beverage container or a medical device.

2. The composition of claim 1, wherein the polymer is an epoxy resin comprising a plurality of hydrogenated bisphenol-A containing units having the formula (I):

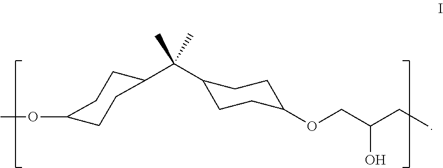

3. The composition of claim 1, wherein the polymer is an epoxy resin which is cross-linked by a polyamine, polyamide, polythiol, or polyol.

4. The composition of claim 1, wherein the polymer is a polyurethane.

5. The composition of claim 4, wherein the polyurethane is cross-linked by an agent selected from the group consisting of methylene-bis(4-cyclohexylisocyanate), 2,2-propylene-bis(4-cyclohexylisocyanate), isophorone diisocyanate, hexamethylene diisocyanate, hexamethylene diisocyanate dimer, hexamethylene diisocyanate trimer, polyisocyanates, toluene diisocyanate, methylene bis(4-phenylisocyanate), benzene diisocyanate, and cyclohexane diisocyanate.

6. The composition of claim 1, wherein the polymer is cross-linked by perfluorocyclobutane linkages.

7. The composition of claim 1, wherein the polymer is a polycarbonate comprising a plurality of hydrogenated bisphenol-A containing units having the formula (II):

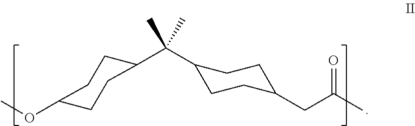

8. (canceled)

9. The composition of claim 7, wherein the polycarbonate further comprises a plurality of cyclohexyl units having the formula (III):

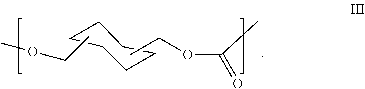

10. The composition of claim 7, wherein the polycarbonate has the formula (V): 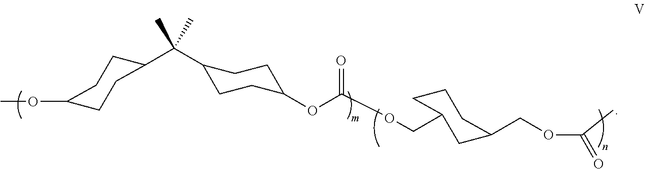
wherein m and n are independently from 2 to 100,000.

11. The composition of claim 7, wherein the polycarbonate further comprises a plurality of bisphenol-A containing units having the formula (VI):

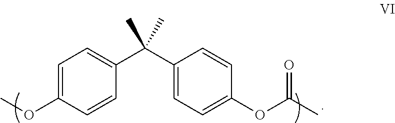

12. (canceled)

13. The composition of claim 1, wherein the polymer is an alkyd resin comprising a polyol backbone, comprising a plurality of hydrogenated bisphenol-A containing units; and
a plurality of fatty acid side chain units attached to the polyol backbone.

14. The composition of claim 13, wherein the fatty acid side chain units comprise one or more of oleic acid, linolenic acid, linoleic acid, eleostearic acid, or palmitic acid.

15. The composition of claim 13, wherein the hydrogenated bisphenol-A containing unit has the formula (VIII): 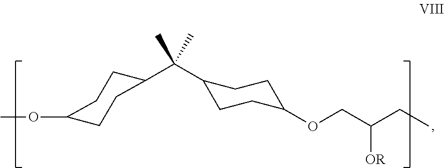
wherein R is H or a fatty acid side chain unit.

16. The composition of claim 13, wherein the fatty acid side chain units comprise from about 80 to about 85% of eleostearic acid, from about 2 to about 6% of oleic acid, from about 3 to about 7% of palmitic acid, and from about 5 to about 10% of linoleic acid by total weight of the side chains.

17. The composition of claim 1, wherein the polymer is a random copolymer, a block copolymer, or a graft copolymer.

18. The composition of claim 1, wherein the polymer is a graft copolymer resin wherein the backbone of the copolymer comprises at least one repeating unit selected from the group consisting of HBPA and HBPA-epichlorohydrin, and the graft side chain is a polyacrylate or a polyolefin.

19. The composition of claim 18 wherein the graft side chain is a polyacrylate comprising at least one repeating unit selected from acrylic acid, acrylic acid esters, methacrylic acid and methacrylic acid esters.

20. The composition of claim 18 wherein the graft side chain is a polyolefin selected from the group consisting of polyethylene, polypropylene, polystyrene and a copolymer of any one thereof.

21. (canceled)

22. A food or beverage container or a medical device, comprising a surface coated with a composition comprising a polymer having at least one repeating unit, wherein the repeating unit is a hydrogenated bisphenol A-containing unit and the polymer is selected from a group consisting of a polycarbonate, an epoxy resin, an alkyd resin, a polyurethane, and a copolymer of any of the foregoing.

23-25. (canceled)

26. A medical device or a food or beverage container, comprising a surface, wherein the surface comprises a composition comprising a polymer having at least one repeating unit, wherein the repeating unit is a hydrogenated bisphenol A-containing unit and the polymer is selected from a group consisting of a polycarbonate, an epoxy resin, an alkyd resin, a polyurethane, and a copolymer of any of the foregoing.
